(19) United States
(12) Patent Application Publication
(10) Pub. No.: US 2007/0122914 A1
(43) Pub. Date: May 31, 2007

(54) Obtaining measurements of light transmitted through an assay test strip

(76) Inventor: Bo U. Curry, Redwood City, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/290,056

(22) Filed: Nov. 30, 2005

(51) Int. Cl. G01N 33/543; C12M 1/34

(52) U.S. Cl. 436/518; 435/287.2

(57) ABSTRACT

Systems and methods of obtaining measurements of light transmitted through an assay test strip are described. In one aspect, a test strip is held. The top side of the test strip is illuminated with light. The illuminating light that is transmitted through the test strip and out from the bottom side of the test strip is detected. In another aspect, a diagnostic test system includes a detection system and a retainer. The detection system includes an optical detector that produces a measurement signal in response to light. The retainer holds a test strip so that the top side of the test strip is exposed for illumination and the bottom side of the test strip faces the optical detector.

### BACKGROUND

Assay test kits currently are available for testing a wide variety of medical and environmental conditions or compounds, such as a hormone, a metabolite, a toxin, or a pathogen-derived antigen. FIG. 1 shows a typical lateral flow test strip 10 that includes a sample receiving zone 12, a labeling zone 14, a detection zone 15, and an absorbent zone 20 on a common substrate 22. These zones 12-20 typically are made of a material (e.g., chemically-treated nitrocellulose) that allows fluid to flow from the sample receiving zone 12 to the absorbent zone 22 by capillary action. The detection zone 15 includes a test region 16 for detecting the presence of a target analyte in a fluid sample and a control region 18 for indicating the completion of an assay test.

FIGS. 2A and 2B show an assay performed by an exemplary implementation of the test strip 10. A fluid sample 24 (e.g., blood, urine, or saliva) is applied to the sample receiving zone 12. In the example shown in FIGS. 2A and 2B, the fluid sample 24 includes a target analyte 26 (i.e., a molecule or compound that can be assayed by the test strip 10). Capillary action draws the liquid sample 24 downstream into the labeling zone 14, which contains a substance 28 for indirect labeling of the target analyte 26. In the illustrated example, the labeling substance 28 consists of an immunoglobulin 30 with a detectable particle 32 (e.g., a reflective colloidal gold or silver particle). The immunoglobulin 30 specifically binds the target analyte 26 to form a labeled target analyte complex. In some other implementations, the labeling substance 28 is a non-immunoglobulin labeled compound that specifically binds the target analyte 26 to form a labeled target analyte complex.

The labeled target analyte complexes, along with excess quantities of the labeling substance, are carried along the lateral flow path into the test region 16, which contains immobilized compounds 34 that are capable of specifically binding the target analyte 26. In the illustrated example, the immobilized compounds 34 are immunoglobulins that specifically bind the labeled target analyte complexes and thereby retain the labeled target analyte complexes in the test region 16. The presence of the labeled analyte in the sample typically is evidenced by a visually detectable coloring of the test region 16 that appears as a result of the accumulation of the labeling substance in the test region 16.

The control region 18 typically is designed to indicate that an assay has been performed to completion. Compounds 35 in the control region 18 bind and retain the labeling substance 28. The labeling substance 28 typically becomes visible in the control region 18 after a sufficient quantity of the labeling substance 28 has accumulated. When the target analyte 26 is not present in the sample, the test region 16 will not be colored, whereas the control region 18 will be colored to indicate that assay has been performed. The absorbent zone 20 captures excess quantities of the fluid sample 24.

Although visual inspection of lateral flow assay devices of the type described above are able to provide qualitative assay results, such a method of reading these types of devices is unable to provide quantitative assay measurements and therefore is prone to interpretation errors. Automated and semi-automated lateral flow assay readers have been developed in an effort to overcome this deficiency. These readers typically include a light source for illuminating the top side of a test strip on which the test and control regions are exposed, and an optical detector that measures light that reflects or fluoresces from the top surface. In these approaches, a significant source of noise is caused by reflection of the illuminating light from non-target surfaces of the test strip and other surfaces within the readers. The noise caused by such stray reflected light may be reduced by increasing the precision with which the test strip is aligned with the optical detector and by performing complex and resource intensive analyses of the measurement data. In general, however, such noise reduction measures increase the cost and complexity of the assay reader design.

What is needed is a diagnostic test system that is capable of obtaining optical measurements from an assay test strip in a way that reduces the susceptibility of the detection system to receive stray reflected light and thereby allows the assay test strips to be evaluated with high accuracy and precision while using relatively inexpensive components and without requiring complex and resource intensive analyses of the measurement data.

### SUMMARY

In one aspect, the invention features a diagnostic test method in accordance with which a test strip is held. The test strip includes a flow path for a fluid sample, a bottom side, and a top side that is opposite the bottom side and that supports a detection zone, which has at least one measurement region coupled to the flow path. The top side of the test strip is illuminated with light. The illuminating light that is transmitted through the test strip and out from the bottom side of the test strip is detected.

In another aspect, the invention features a diagnostic test system that includes a detection system and a retainer. The detection system includes an optical detector that produces a measurement signal in response to light. The retainer holds a test strip so that the top side of the test strip is exposed for illumination and the bottom side of the test strip faces the optical detector.

Other features and advantages of the invention will become apparent from the following description, including the drawings and the claims.

### DESCRIPTION OF DRAWINGS

FIG. 1 is a diagrammatic view of a prior art implementation of an assay test strip.

FIG. 2A is a diagrammatic view of a fluid sample being applied to a sample receiving zone of the assay test strip shown in FIG. 1.

FIG. 2B is a diagrammatic view of the assay test strip shown in FIG. 2A after the fluid sample has flowed across the test strip to an absorption zone.

FIG. 3 is a diagrammatic side view of an embodiment of a diagnostic test system that includes a detection system and a retainer that is holding an assay test strip.

FIG. 4 is a diagrammatic side view of the diagnostic test system shown in FIG. 3 in which the detection system is obtaining intensity measurements of light passing through the test strip before an assay has been performed.

FIG. 5 is a simulated graph of the transmitted light intensity measured by the detection system with the test strip in the state shown in FIG. 4 plotted as a function of position along the test strip.

FIG. 6 is a diagrammatic side view of the diagnostic test system shown in FIG. 3 in which the detection system is obtaining intensity measurements of light passing through the test strip after an assay has been performed.

FIG. 7 is a simulated graph of the transmitted light intensity measured by the detection system with the test strip in the state shown in FIG. 6 plotted as a function of position along the test strip.

FIG. 8 is a simulated graph of the difference between the light intensity measurements shown in FIGS. 5 and 7 plotted as a function of position along the test strip.

FIG. 9 is a diagrammatic side view of an embodiment of the diagnostic test system shown in FIG. 3 in which the detection system includes two optical detectors.

FIG. 10 is a diagrammatic side view of an embodiment of the diagnostic test system shown in FIG. 3 that is incorporated within a housing that includes an optically transparent window for illuminating the top side of the test strip with external ambient light.

FIG. 11 is a diagrammatic side view of an embodiment of the diagnostic test system shown in FIG. 3 that is incorporated within a housing that includes a light source that illuminates the top side of the test strip.

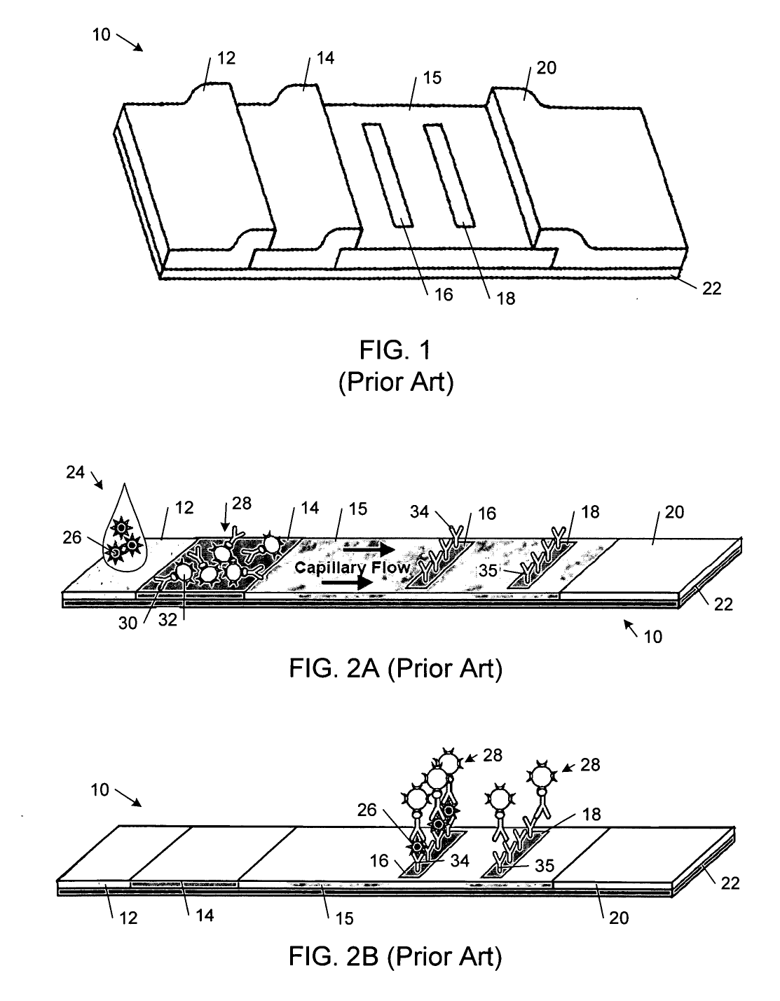

### DETAILED DESCRIPTION

In the following description, like reference numbers are used to identify like elements. Furthermore, the drawings are intended to illustrate major features of exemplary embodiments in a diagrammatic manner. The drawings are not intended to depict every feature of actual embodiments nor relative dimensions of the depicted elements, and are not drawn to scale.

I. Introduction

The embodiments that are described herein provide systems and methods of obtaining measurements of light transmitted through assay test strips and using these measurements to evaluate assays performed on the assay test strips. The light that is transmitted through the test strips is substantially free of light that is reflected from non-target surfaces of the test strips and other surfaces within the diagnostic test system. For this reason, the alignment constraints between the test strips and the detection system in the embodiments that are described below may be reduced relative to diagnostic test approaches that measure light from the illuminated surfaces of the test strips. The need for complex and resource intensive analyses of the measurement data in order to reduce the noise caused by reflected light also is reduced. These features allow the embodiments that are described herein to evaluate assay test strips with high accuracy and precision while using relatively inexpensive components and without requiring complex and resource intensive analyses of the measurement data.

The terms “assay test strip” and “lateral flow assay test strip” encompass both competitive types of assay test strips in which an increase in the concentration of the analyte in the sample results in an increase in the concentration of labels in the test region and non-competitive types of assay test strips in which an increase in the concentration of the analyte in the fluid sample results in a decrease in the concentration of labels in the test region. A lateral flow assay test strip generally includes a sample receiving zone and a detection zone, and may or may not have a labeling zone. In some implementations, a lateral flow assay test strip includes a sample receiving zone that is located vertically above a labeling zone, and additionally includes a detection zone that is located laterally downstream of the labeling zone.

The term “analyte” refers to a substance that can be assayed by the test strip. Examples of different types of analytes include organic compounds (e.g., proteins and amino acids), hormones, metabolites, antibodies, pathogen-derived antigens, drugs, toxins, and microorganisms (e.g., bacteria and viruses).

As used herein the term “label” refers to a substance that has specific binding affinity for an analyte and a detectable characteristic feature that can be distinguished from other elements of the test strip. The label may include a combination of a labeling substance (e.g., a fluorescent particle, such as a quantum dot) that provides the detectable characteristic feature and a probe substance (e.g., an immunoglobulin) that provides the specific binding affinity for the analyte. In some implementations, the labels have distinctive optical properties, such as luminescence (e.g., fluorescence) or reflective properties, which allow regions of the test strip containing different labels to be distinguished from one another.

The term “reagent” refers to a substance that reacts chemically or biologically with a target substance, such as a label or an analyte.

The term “capture region” refers to a region on a test strip that includes one or more immobilized reagents.

The term “test region” refers to a capture region containing an immobilized reagent with a specific binding affinity for an analyte.

The term “control region” refers to a capture region containing an immobilized reagent with a specific binding affinity for a label.

The term “measurement region” refers to any region of interest on an assay test strip, such as a capture region or a calibration region, that may be measured for the purpose of evaluating the assay test strip. The term “baseline region” refers to any region of an assay test strip outside of a measurement region.

The term “measurement signal” refers to a signal that is produced by an optical detector in response to light received from a measurement region of an assay test strip. The term “baseline signal” refers to a signal that is produced by an optical detector in response to light received from a baseline region of an assay test strip.

The phrase “quantifying a first value with respect to a second value” refers to a process of deriving a final quantified value from a function that compares the first and second values or that compares values that are derived respectively from the first and second values. The comparison function may include a ratio between the first and second values, a difference between the first and second values, or some other mathematical function of the first and second values.

II. General Diagnostic Test System Architecture

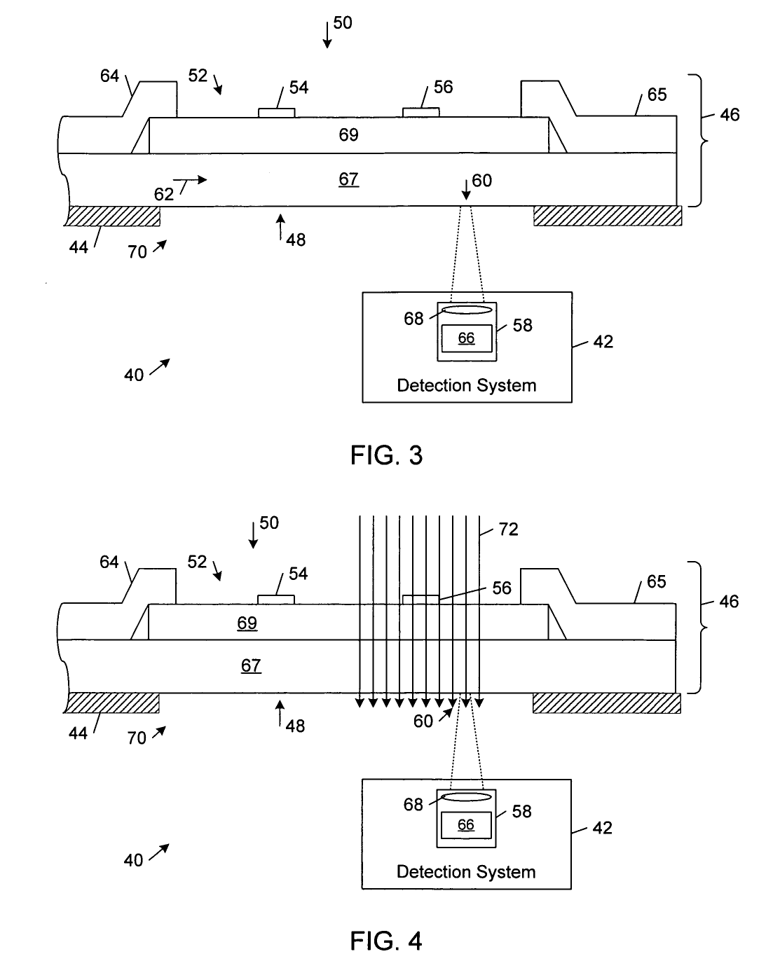

FIG. 3 shows an embodiment of a diagnostic test system 40 that includes a detection system 42 and a retainer 44 that holds a test strip 46. The test strip 46 has a bottom side 48 and a top side 50 that is opposite the bottom side 48 and supports a detection zone 52, which includes a test region 54 and a control region 56. The detection system 42 includes an optical detector 58 that has a field of view 60 that corresponds to an area on the bottom side 48 of the test strip 46. When the test strip 46 is held by the retainer 44, the optical detector 58 obtains intensity measurements of light that passes from a region above the top side 50 of the test strip 46, through the test strip 46, and out the bottom side 48 of the test strip 46. The light intensity measurements typically are transmitted to a data analyzer (not shown in FIG. 3), which computes at least one parameter from one or more of the light intensity measurements. A results indicator (not shown in FIG. 3) typically provides an indication of one or more of the results of an assay of the test strip 46. In some implementations, the diagnostic test system 40 is fabricated from relatively inexpensive components enabling it to be used for disposable or single-use applications.

In the illustrated embodiments, each of the test strips 46 is a non-competitive type of assay test strip that supports lateral flow of a fluid sample along a lateral flow direction 62. Each of the test strips 46 includes a labeling zone 64, a detection zone 52, and an absorbent zone 65 that are formed on a common substrate 67. The labeling zone 64 contains a labeling substance that binds a label to a target analyte that may be present in a fluid sample to be assayed. The detection zone 52 includes a membrane 69 that supports at least one test region 54 containing an immobilized substance that binds the target analyte and at least one control region 56 containing an immobilized substance that binds the label. One or more areas of the detection zone 52, including at least a portion of the test region 54 and the control region 56, are exposed for illumination from a region above the top side 50 of the test strip 46. The source of the illumination may be ambient light or an active light source, such as a light emitting diode.

The membrane 69 and the substrate 67 are formed of respective materials that are translucent with respect to light within a target wavelength range (e.g., visible light or infrared light) that is detectable by the optical detector 58. Exemplary materials from which the membrane 69 and the substrate may be formed include nitrocellulose (cellulose nitrate), paper, glass fibers, polypropylene, and cellulose acetate. In some embodiments, the various zones 52, 64, and 65 may be formed on a single translucent sheet of material and the substrate 67 may be omitted. In other embodiments, the substrate 67 may include one or more windows that are aligned vertically (i.e., orthogonally with respect to the top and bottom sides 50, 48 of the test strip) with respective measurement and baseline regions of the detection zone 52.

In other embodiments, the test strips 46 are competitive type of lateral flow assay test strips in which the concentration of the label in the test region decreases with increasing concentration of the target analyte in the fluid sample. Some of these embodiments include a labeling zone, whereas others of these implementations do not include a labeling zone.

Some of these competitive lateral flow assay test strip embodiments include a labeling zone that contains a label that specifically binds target analytes in the fluid sample, and a test region that contains immobilized target analytes as opposed to immobilized test reagents (e.g., antibodies) that specifically bind any non-bound labels in the fluid sample. In operation, the test region will be labeled when there is no analyte present in the fluid sample. However, if target analytes are present in the fluid sample, the fluid sample analytes saturate the label's binding sites in the labeling zone, well before the label flows to the test region. Consequently, when the label flows through the test region, there are no binding sites remaining on the label, so the label passes by and the test region remains unlabeled.

In other competitive lateral flow assay test strip embodiments, the labeling zone contains only pre-labeled analytes (e.g., gold adhered to analyte) and the test region contains immobilized test reagents with an affinity for the analyte. In these embodiments, if the fluid sample contains unlabeled analyte in a concentration that is large compared to the concentration of the pre-labeled analyte in the labeling zone, then label concentration in the test region will appear proportionately reduced.

The detection system 46 includes one or more optoelectronic components for optically inspecting the exposed areas of the detection zone of the test strip 50. In some implementations, the detection system 46 includes at least one optical detector 58, which includes a respective optoelectronic transducer 66. The optoelectronic transducer 66 produces electrical measurement signals in response to receipt of light that is transmitted through the test strip 46. The optoelectronic transducer 66 may be implemented by any type of photodetector device, including a one-dimensional optical detector (e.g., a photodiode device) and a two-dimensional optical detector (e.g., a CCD or CMOS image sensor device). The optical detector 58 also may include an optical system 68 that guide light from the bottom side 50 of the test strip 46 onto the respective active areas of the optoelectronic transducer 66. The optical system 68 may include one or more optical elements (e.g., refractive lenses, diffractive lenses, and optical filters) that intercept and modify the light received from respective regions of the bottom side 50 of the test strip 46.

In some implementations, the optical detector 58 may be designed to selectively capture light that is transmitted though the test strip 46. For example, the optical detector 58 may be designed to selectively capture light corresponding to the light illuminating the top side 50 of the test strip 46. For example, if the illuminating light is provided by a light source that produces light within a specified wavelength range or that has a specified polarization, the optical detector 58 may be designed to selectively capture light within the specified wavelength range or having the specified polarization. In these embodiments, the optical detector 58 may include one or more optical filters that define the wavelength ranges or polarizations axes of the detected light.

The retainer 44 holds the test strip 46 so that the top side 50 is exposed for illumination and the bottom side 48 is faced by the optical detector 58. In some embodiments, the retainer 44 is configured to move the test strip 46 relative to the detection system 42. In other embodiments, the detection system 42 is configured to move relative to the test strip 46. In some embodiments, both the retainer 44 and the detection system 42 move relative to each other. In some embodiments, the movable ones of the retainer 44 and the detection system 42 are moved manually by a user of the diagnostic test system 40. Other embodiments of the diagnostic test system 40 include at least one motor that moves the movable ones of the retainer 44 and the detection system 42. These embodiments typically include a position encoder (e.g., an optical encoder) that produces signals that track the relative position of the retainer 44 with respect to the detection system 42. These embodiments also typically include a data analyzer that is operable to correlate the measurements obtained by the detection system 42 with locations along the test strip 46 based on the signals produced by the position encoder.

As shown in FIG. 3, the retainer 44 includes a window 70 that allows light that is transmitted through the test strip 46 to reach the optical detector 58 when the test strip 46 is held by the retainer 44. In some implementations, the window 70 may consist of an opening in the support structure of the retainer 44, as shown in FIG. 3. In other implementations, the window 70 may include a material that is optically transparent to light within a target wavelength range (e.g., visible light or infrared light) that is detectable by the optical detector 58. In some implementations, the retainer 44 is formed of a material (e.g., glass or quartz) that is optically transparent to light within the target wavelength range.

III. Obtaining Measurement Signals for Evaluating Assay Test Strips

FIG. 4 shows the diagnostic test system 40 and the test strip 46 in a state before an assay has been performed. In this state, the test region 54 and the control region 56 have not immobilized any of the label from the labeling zone 64. Consequently, the transmittance (i.e., the ratio of the transmitted optical power to the incident optical power) of light 72 through the thickness of the test strip 46 at the locations of the test and control regions 54, 56 will correspond to the transmittance through the test strip 46 at other locations in the detection zone 52, except for attenuations that might be caused by the presence of the immobilized substances in the test and control regions 54, 56 that bind the target analyte and the label, respectively. In typical implementations of the test strip 46, however, the attenuation of the transmitted light 72 by the immobilized substances is expected to be substantially smaller than the light attenuation that is caused by the presence of the label in the test and control regions 54, 56. In some embodiments, one or more of the properties of the illuminating light 72 and the immobilized substances may be selected so that the attenuation of the light 72 caused by the presence of the immobilized substances the test and control regions 54, 56 is substantially smaller than the light attenuation that is caused by the presence of the label in the test and control regions 54, 56.

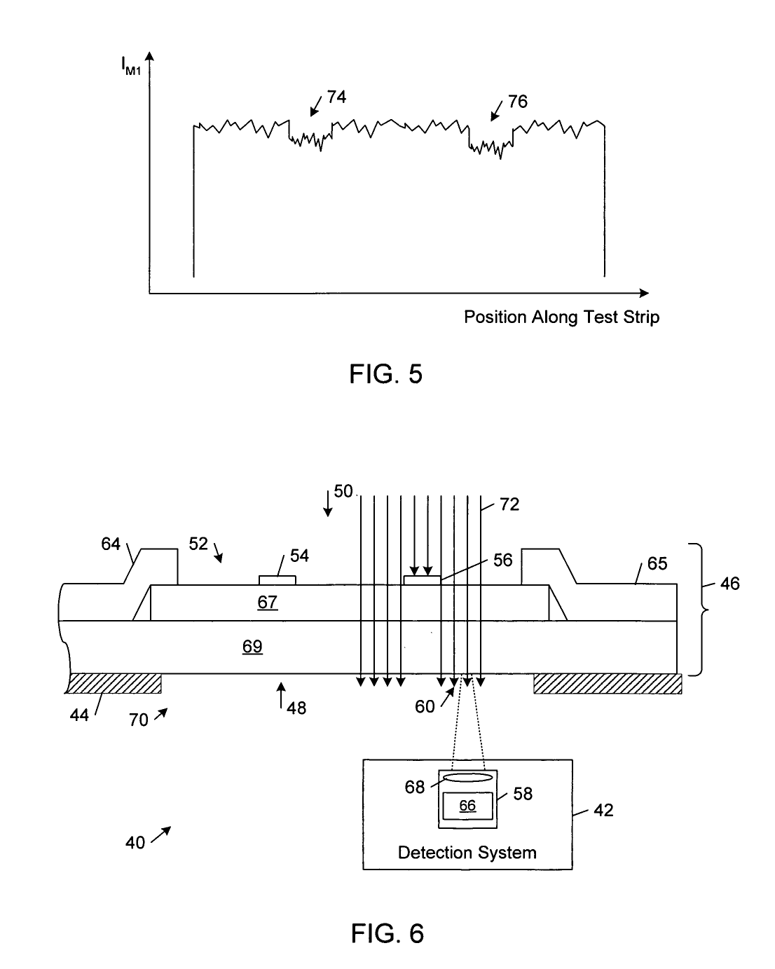

FIG. 5 shows an exemplary simulated graph of the transmitted light intensity measured by the optical detector 58 with the test strip 46 in the state shown in FIG. 4 plotted as a function of position along the test strip 46. In this example, the light intensity measured by the optical detector 58 is substantially uniform across the detection zone 52, except in the positions 74, 76 corresponding to the test and control regions 54, 56. At these positions 74, 76, the graph is intended to show the relatively small reductions in the transmitted light intensity that are expected to be caused by the immobilized substances in the test and control regions 54, 56 of the detection zone 52.

FIG. 6 shows the diagnostic test system 40 and the test strip 46 in a state after an assay has been performed. In this state, the test region 54 will contain ones of the labeling compounds that are bound to the target analyte in the fluid sample that is the subject of the assay. In addition, the control region 56 will contain excess ones of the labeling compounds that are transported from the labeling zone 64 by the capillary migration of the fluid sample across the detection zone 52. In this state, the transmittance of light 72 through the thickness of the test strip 46 at locations corresponding to the test and control regions 54, 56 will not correspond to the transmittance through the thickness of the test strip 46 at other locations of the detection zone 52 due to the presence of the immobilized label in the test and control regions 54, 56. In particular, the presence of the label in the test and control regions 54, 56 blocks or substantially reduces the intensity of illuminating light that is transmitted through the test strip 46, as shown diagrammatically in FIG. 6.

In some embodiments, the label is a reflective label that reflects the illuminating light 72 away from the top side 50 of the test strip 46. For example, some labels (e.g., colloidal gold and silver particles) have reflectivities that are greater than 90% with respect to light within a target wavelength range corresponding to visible light (i.e., 390 nm to 770 nm). In other embodiments, the label is an absorptive label that blocks light from being transmitted through the test strip 46. For example, some labels (e.g., quantum dots) may absorb the illuminating light within a target wavelength range and emit secondary fluorescent light at longer wavelengths. In these embodiments, the intensities of the secondary fluorescent emissions that may be detected by the optical detector 58 are expected to be substantially lower than the intensity of the primary illuminating light 72 that is transmitted through the test strip 46.

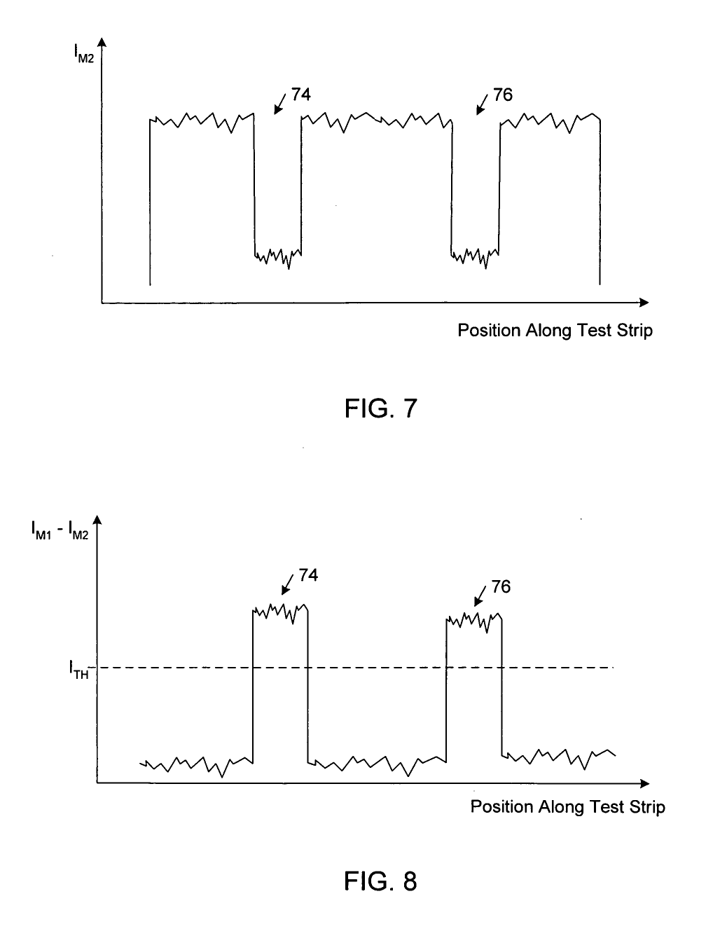

FIG. 7 is an exemplary simulated graph of the transmitted light intensity measured by the optical detector 58 with the test strip 46 in the state shown in FIG. 6 plotted as a function of position along the test strip 46. In this example, the light intensity measured by the optical detector 58 is substantially uniform across the detection zone 52, except in the positions 74, 76 corresponding to the locations of the test and control regions 54, 56. At these positions 74, 76, the graph is intended to show the relatively large reductions in the transmitted light intensity that are expected to be caused by the presence of the label in the test and control regions 54, 56 of the detection zone 52.

FIG. 8 shows an exemplary simulated graph of the intensity difference (I_{M1}-I_{M2}) between the light intensity measurements (I_{M1} and I_{M2}) shown in FIGS. 5 and 7 plotted as a function of position along the test strip. The intensity difference graph shows peaks in the positions 74, 76 along the test strip 46 corresponding to the locations of the test and control regions 74, 76. In this example, the slight reductions in the transmitted light intensities that were caused by the presence of the immobilized substances in the test and control regions 54, 56 have only insubstantial effects on the intensity difference graph shown in FIG. 8. In some embodiments, an empirically determined threshold (I_{TH}) is applied to the intensity difference graph to identify the presence of the label in the test and the control regions 54, 56. In these embodiments, the presence of the label is detected if the intensity difference is greater than the threshold. In some embodiments, the presence of the labels in the test and control regions 54, 56 is determined by application different thresholds to the portions 74, 76 of the intensity difference graph corresponding to the locations of the test and control regions 54, 56.

IV. Exemplary Implementations of the Diagnostic Test System

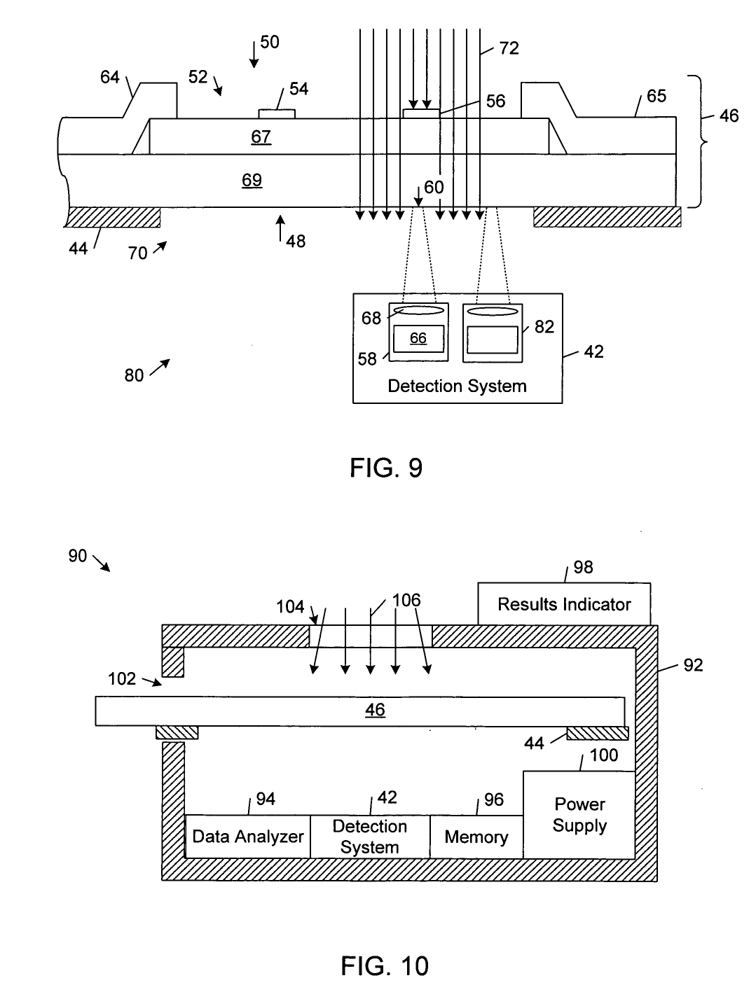

FIG. 9 shows an embodiment 80 of the diagnostic test system 40 in which the detection system 42 includes a first optical detector 58 and a second optical detector 82. In the illustrated embodiment, the second optical detector 82 is implemented in the same way as the first optical detector 58. In other embodiments, the second optical detector 82 may include different components or a different configuration of the same components as the first optical detector 82.

In the diagnostic test system embodiment 80, the first optical detector 58 produces measurement signals in a measurement data channel in response to the receipt of light that is transmitted through the test strip 46 in areas of the bottom side 48 that are aligned with (or shadowed by) measurement regions of the test strip 46. The second optical detector 82 produces baseline signals in a baseline data channel separate from the measurement data channel in response to the receipt of light that is transmitted through the test strip in areas of the bottom side 48 of the test strip 46 that are aligned with respective regions of the test strip that are outside of any measurement region. Producing the measurement and baseline signals in separate data channels allows the assay test strip 46 to be evaluated with high accuracy and precision while using relatively inexpensive detectors and processing components.

In some embodiments, for each measurement region in the detection zone 15, at least one of the detection system 42 and the retainer 44 are moved into a respective measurement position in which the test strip 46 is aligned vertically (i.e., orthogonally to the top and bottom sides 50, 48 of the test strip 46) with the detection system 42 such that the first optical detector 58 is positioned directly under an area of the bottom side 48 of the test strip that corresponds to a measurement region of the detection zone 52. For example, FIG. 9 shows the test strip 46 and the detection system 42 in a first measurement position in which the first optical detector 58 is positioned directly under the control region 56 and the second optical detector 82 is positioned directly under a baseline region of the detection zone 15 that is adjacent to the control region 18 but outside of any measurement region (i.e., the test region 54 and the control region 56). In a second measurement position, the first optical detector 58 would be positioned directly under the test region 54 and the second optical detector 82 would be positioned directly under a baseline region of the detection zone 15 that is adjacent to the test region 54 but outside of the test region 54 and the control region 56.

Some embodiments of the diagnostic test system 40 may include an alignment system that is configured cooperatively with the detection system 42 to guide the movement of at least one of the retainer 44 and the detection system 42 into a respective measurement position in which the test strip 46 is aligned with the detection system 42 for each measurement region in the detection zone 15. In each measurement position, the first optical detector 58 receives light predominantly from an area of the bottom side 48 of the test strip that is aligned vertically with a respective one of the measurement regions and the second optical detector 82 receives light predominantly from an area of the bottom side 48 of the test strip that is aligned vertically with baseline region outside of any measurement region.

FIG. 10 shows an embodiment 90 of the diagnostic test system 40 that includes a housing 92, the detection system 42, a data analyzer 94, a memory 96, a results indicator 98, and a power supply 100. The housing 92 includes a port 102 for receiving the test strip 46 and a window 104 for illuminating the test strip 46 with external light 106 (e.g., ambient light). The window 104 may consist of an opening in the housing 92 or it may include a material that is optically transparent to ambient light 106 within a target wavelength range (e.g., visible light or infrared light). In some implementations, the diagnostic test system 90 is fabricated from relatively inexpensive components enabling it to be used for disposable or single-use applications.

The housing 92 may be made of any one of a wide variety of materials, including plastic and metal. The housing 92 forms a protective enclosure for the detection system 42, the retainer 44, the data analyzer 94, the memory 96, the power supply 100, and other components of the diagnostic test system 90. The housing 92 also may include the above-described alignment system, which mechanically registers the test strip 46 with respect to the detection system 42.

The results indicator 98 may include any one of a wide variety of different mechanisms for indicating one or more results of an assay test. In some implementations, the results indicator 98 includes one or more lights (e.g., light-emitting diodes) that are activated to indicate, for example, a positive test result and the completion of the assay test (i.e., when sufficient quantity of the labeling substance has accumulated in the control region). In other implementations, the results indicator 98 includes an alphanumeric display (e.g., a two or three character light-emitting diode array) for presenting assay test results.

The power supply 100 supplies power to the active components of the diagnostic test system 90, including the detection system 42, the data analyzer 94, and the results indicator 98. The power supply 100 may be implemented by, for example, a replaceable battery or a rechargeable battery. In other embodiments, the diagnostic test system may be powered by an external host device (e.g., a computer connected by a USB cable).

The data analyzer 94 processes the light intensity measurements that are obtained by the detection system 42. In general, the data analyzer 94 may be implemented in any computing or processing environment, including digital electronic circuitry or computer hardware, firmware, or software. In some embodiments, the data analyzer 94 includes a processor (e.g., a microcontroller, a microprocessor, or ASIC) and an analog-to-digital converter. In the illustrated embodiment, the data analyzer 94 is incorporated within the housing 92 of the diagnostic test system 90. In other embodiments, the data analyzer 94 is located in a separate device, such as a computer, that may communicate with the diagnostic test system 90 over a wired or wireless connection.

In operation, the test strip 46 is loaded onto the retainer 44 and moved into the port 102. The detection system 42 obtains intensity measurements of light passing through the test strip 46. In particular, the detection system 42 produces measurement signals in response to light received from respective areas of the bottom side 48 of the test strip 46 that are aligned with respective measurement regions of the detection zone 52. The detection system 42 also produces baseline signals in response to light received from respective areas of the bottom side 48 of the test strip 46 that are aligned with baseline regions of the detection zone 52.

The measurement and baseline signals may be obtained by a single optical detector or by multiple optical detectors, as explained above. The data analyzer 94 computes at least one parameter from one or more of the light intensity measurements. In particular, the data analyzer 94 quantifies the respective ones of the measurement signals with respect to respective ones of the baseline signals for each measurement position of the test strip 46 relative to the detection system 42. In this process, the data analyzer 94 derives a final quantified value from a function that compares the measurement region values and baseline region values. The measurement region value is derived from the measurement signals (e.g., an average or a peak signal value) and optionally may be calibrated with respect to a dark value that is measured when the top side 50 of the test strip 46 is not illuminated (e.g., when the light source is turned off). Similarly, the baseline region value is derived from the baseline signals (e.g., an average or a peak signal value) and optionally may be calibrated with respect to a dark value that is measured when the top side 50 of the test strip 46 is not illuminated.

The comparison function may include a ratio between the measurement region value and the baseline region value, a difference between the measurement region value and baseline region value, or some other mathematical function of the measurement region value and the baseline region value. For example, in some embodiments, the data analyzer 94 quantifies the measurement region value in terms of the baseline region value to determine a measure of the transmission density of a respective one of the measurement regions of the test strip 46. The transmission density is the logarithm of the transmittance to the base 10, where the transmittance is the ratio of the measurement region value to the baseline region value. The data analyzer 94 may use the transmission density value as an index into a lookup table that maps transmission density values to analyte concentration values.

The results indicator 98 provides an indication of one or more of the results of an assay of the test strip 46 based on the parameters that are computed by the data analyzer 94.

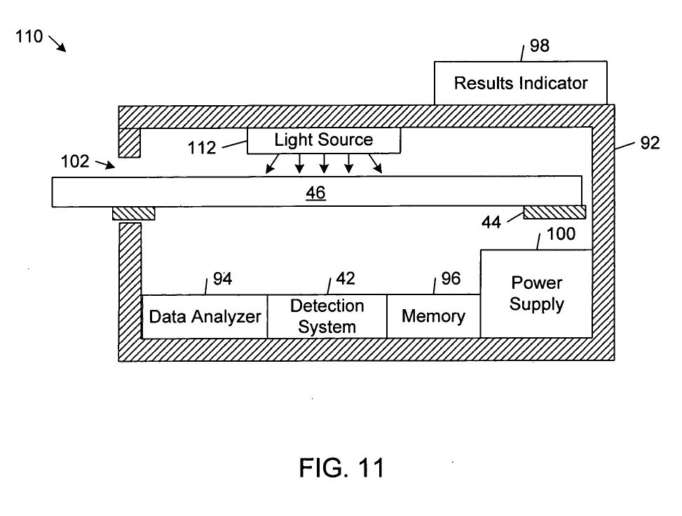

FIG. 11 shows an embodiment 110 of the diagnostic test system 40 that corresponds to the embodiment 80 that is shown in FIG. 10, except that the window 104 is replaced by a light source 112 that is configured to illuminate the top side 50 of the test strip 46 with light within a target wavelength range (e.g., visible light or infrared light). In some implementations, the light source 112 includes a semiconductor light-emitting diode. Depending on the nature of the label that is used by the test strip 46, the light source 112 may be a broadband light source or it may be designed to emit light within a particular wavelength range or with a particular polarization, in which case the light source 112 may include one or more optical filters that define the wavelength ranges or polarizations axes of the light.

Some embodiments of the diagnostic test system 40 include one or more of the systems for aligning the detection system with the assay test strip. These alignment systems may be used by the data analyzer 94 to correlate the measurement and baseline signals that are produced by the detection system 42 with positions along the test strip 46, which enables the data analyzer 94 to associate the measurement signals with the corresponding measurement regions in the detection zone.

### V. CONCLUSION

The embodiments that are described in detail above provide systems and methods of obtaining measurements of light transmitted through assay test strips and using these measurements to evaluate assays performed on the assay test strips. The light that is transmitted through the test strips is substantially free of light that is reflected from non-target surfaces of the test strips and other surfaces within the diagnostic test system. For this reason, the alignment constraints between the test strips and the detection system in the embodiments that are described below may be reduced relative to diagnostic test approaches that measure light from the illuminated surfaces of the test strips. The need for complex and resource intensive analyses of the measurement data in order to reduce the noise caused by reflected light also is reduced. These features allow the embodiments that are described above to evaluate assay test strips with high accuracy and precision while using relatively inexpensive components and without requiring complex and resource intensive analyses of the measurement data.

Other embodiments are within the scope of the claims.

1. (canceled)

2. A diagnostic test system, comprising:
a detection system comprising a first optical detector operable to produce a measurement signal in response to light;
a retainer configured to hold a test strip comprising a flow path for a fluid sample, a bottom side, and a top side opposite the bottom side and supporting a detection zone that has at least one measurement region coupled to the flow path, wherein the retainer is configured to hold the test strip so that the top side is exposed for illumination and the bottom side faces the first optical detector; and
a housing containing the detection system and the retainer and comprising a translucent window permitting light from outside the housing to illuminate the measurement region when the test strip is held by the retainer.

3. (canceled)

4. The system of claim 2, wherein the detection system comprises a second optical detector facing the bottom side of the test strip when the test strip is held by the retainer and operable to produce a measurement signal in response to light.

5. The system of claim 4, wherein the first and second optical detectors have respective fields of view containing different respective areas of the bottom side of the test strip when the test strip is held by the retainer.

6. A diagnostic test system, comprising:
a detection system comprising first and second optical detectors each operable to produce respective measurement signals in response to light;
a retainer configured to hold a test strip comprising a flow path for a fluid sample, a bottom side, and a top side opposite the bottom side and supporting a detection zone that has multiple measurement regions coupled to the flow path, wherein the retainer is configured to hold the test strip so that the top side is exposed for illumination and the bottom side faces the optical detector, wherein
the second optical detector faces the bottom side of the test strip when the test strip is held by the retainer,
the first and second optical detectors have respective fields of view containing different respective areas of the bottom side of the test strip when the test strip is held by the retainer, and
the retainer is configured to hold the test strip in multiple measurement positions relative to the detection system, wherein in each of the measurement positions the field of view of the first optical detector corresponds to an area of the bottom side of the test strip shadowed by a respective one of the measurement regions and the field of view of the second optical detector corresponds to an area of the bottom side of the test strip free of shadowing by any of the measurement regions; and
a data analyzer operable to quantify respective ones of the measurement signals produced by the first optical detector with respect to ones of the measurement signals produced by the second optical detector for each of the measurement positions.

7. The system of claim 5, wherein the fields of view of the first and second optical detectors correspond to respective areas of the bottom side of the test strip displaced from one another in a direction parallel to the flow path.

8. The system of claim 4, further comprising a data analyzer operable to quantify respective ones of the measurement signals produced by the first optical detector with respect to ones of the measurement signals produced by the second optical detector.

9. The system of claim 8, wherein the data analyzer is operable to derive a quantified value by evaluating a function that compares respective ones of the measurement signals produced by the first optical detector and ones of the measurement signals produced by the second optical detector.

10. The system of claim 2, wherein the retainer comprises a window that allows light that is transmitted through the test strip to reach the optical detector when the test strip is held by the retainer.

11. The system of claim 2, further comprising the test strip.

12. The system of claim 11, wherein the test strip is translucent with respect to light within a wavelength range detectable by the optical detector.

13. The system of claim 12, wherein the test strip comprises a labeling zone containing a labeling substance that binds a label to a target analyte, and at least one of the at least one measurement region contains an immobilized reagent that binds the target analyte, wherein the label absorbs or reflects light within the wavelength range.

14. The system of claim 2, further comprising a motor operable to move at least one of the detection system and the retainer relative to one another, a position encoder operable to produce a signal that tracks relative movement between the detection system and the retainer, and a data analyzer operable to correlate the measurement signal with at least one position along the test strip based on the signal produced by the position encoder.

15-21. (canceled)

22. The system of claim 6, further comprising an alignment system configured cooperatively with the detection system to guide movement of at least one of the retainer and the detection system into each of the measurement positions.

23. The system of claim 6, wherein the fields of view of the first and second optical detectors correspond to respective areas of the bottom side of the test strip displaced from one another in a direction parallel to the flow path.

24. The system of claim 6, further comprising a light source operable to illuminate the measurement region when the test strip is held by the retainer.

25. The system of claim 6, wherein the data analyzer is operable to derive a quantified value by evaluating a function that compares respective ones of the measurement signals produced by the first optical detector and ones of the measurement signals produced by the second optical detector.

26. The system of claim 6, wherein the retainer comprises a window that allows light that is transmitted through the test strip to reach the optical detector when the test strip is held by the retainer.

27. The system of claim 6, further comprising the test strip, wherein the test strip is translucent with respect to light within a wavelength range detectable by the optical detector, wherein the test strip comprises a labeling zone containing a labeling substance that binds a label to a target analyte, and at least one of the at least one measurement region contains an immobilized reagent that binds the target analyte, wherein the label absorbs or reflects light within the wavelength range.

29. The system of claim 6, further comprising a motor operable to move at least one of the detection system and the retainer relative to one another, a position encoder operable to produce a signal that tracks relative movement between the detection system and the retainer, and a data analyzer operable to correlate the measurement signal with at least one position along the test strip based on the signal produced by the position encoder.
